(19) United States
(12) Patent Application Publication
(10) Pub. No.: US 2004/0219622 A1
(43) Pub. Date: Nov. 4, 2004

(54) Phosphine-containing formulations for chemiluminescent luciferase assays

(76) Inventor: M. Dean Savage, Rockford (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/495,203

(22) PCT Filed: Nov. 18, 2002
(86) PCT No.: PCT/US02/36905

Related U.S. Application Data
(60) Provisional application No. 60/332,843, filed on Nov. 16, 2001.

(51) Int. Cl. C12Q 1/66; G01N 21/76

(52) U.S. Cl. 435/8; 422/52

(57) ABSTRACT

A composition for the chemiluminescent assay of the activity of a luciferase comprising (i) a substrate for the luciferase, which, upon enzymatic reaction with the luciferase in the presence of any required cosubstrate and/or cofactor, yields a detectable chemiluminescent singal, (ii) any cosubstrate and/or cofactor required or beneficial for enzymatic activity of the luciferase, and, as an improvement of the composition, (iii) a water-soluble, organic phosphine-containing compound, which enables the light output from the enzymatic reaction to be modulated. The composition can be used in applications designed to quantitate the presence of the enzyme(s) itself, either in single or dual format, or in applications designed to quantitate the presence of a required cosubstrate, such as ATP.

### TECHNICAL FIELD OF THE INVENTION

The present invention relates to a composition useful in the chemiluminescent assay of the activity of a luciferase, especially firefly and Renilla luciferases, and more particularly, to an improved chemiluminescent assay for these enzymes. In yet another respect, this invention provides an improved chemiluminescent assay for quantification of a cosubstrate, such as adenosine triphosphate (ATP).

### BACKGROUND OF THE INVENTION

A variety of beetles are known to have bioluminescent properties. Perhaps, the most widely studied beetle has been the American firefly, Photinus pyralis. This beetle possesses a luciferase (E.C. 1.13.12.7), which is capable of producing a green flash of light with a peak emission at approximately 560 nm in an aqueous solution in the presence of substrates for the enzyme and the enzyme's cofactors, which, for some enzymes, beneficially support, or are necessary to support, the enzymatic reaction with the substrate(s).

For firefly luciferase, a chemiluminescent substrate is 4,5-dihydro-2-[6-hydroxy-2-benzothiazolyl]-4-thiazolecarboxylic acid (luciferin), preferably the D-isomer. Cosubtrates, such as a source of high-energy phosphate (e.g., ATP) and oxygen, are required and are consumed in the enzymatic reaction. For firefly luciferase, the presence of a cofactor, e.g., Mg²⁺, is required to support the chemiluminescent reaction. Coenzyme A (CoA) also can be present as a stimulatory cofactor. See, e.g., Anal. Biochem. 219: 169-184 (1994) for a review of the luciferase reaction.

Firefly luciferase is important in the construction of gene reporter assays. For example, the gene encoding firefly luciferase can be inserted into a mammalian cell in combination with a gene of interest to be studied and made to respond to the same regulatory controls as that of the gene of interest. Gene expression, or the absence thereof, then can be determined by assaying the enzymatic activity of the expressed luciferase in the presence of luciferin as a substrate. In such fashion, regulatory elements of gene expression can be studied.

The biochemical aspects of the reactions of firefly luciferase in the presence of its substrates have been studied extensively. The enzyme contains an essential thiol group required for activity (DeLuca et al., Biochemistry 3: 935 (1964); and Lee et al., Biochemistry 8: 130-136 (1969)). Assay compositions for luciferase activity utilizing dithiothreitol (DTT) as a protective reagent have been described (Leach et al., Methods in Enzymology, 133: 51-70 (1986) at page 58, second paragraph; Hall et al., J. Biolum. Chemilum. 2: 41-44 (1988); and Webster et al., J. Appl. Biochem. 2: 469-479 (1980)). The stimulatory effect of CoA on the activity of firefly luciferase was first noted by Airth et al. (Biochimica et Biophysica Acta 27: 519-532 (1958)).

U.S. Pat. Nos. 5,283,179; 5,650,289 and 5,641,641 describe compositions for firefly luciferase assays that employ concentrations of DTT higher than those illustrated in the above article and also show compositions containing stimulatory levels of CoA. The illustrated concentrations of thiols are reported to contribute to enzyme stability during catalysis. While initial light output is extended, there is signal decay after 5 minutes of reaction. The patents also note that the surfactant, Triton X-100, increases initial light output, but is followed by an increased rate of decay. Other nonionic surfactants were reported to have little effect on enzyme activity.

U.S. Pat. No. 5,618,682 illustrates compositions for the firefly luciferase assay which contain adenosine monophosphate (AMP) in conjunction with DTT. Prolonged light output is reported, with the light output decaying in a linear fashion with a half-life of 3.5 to 5 hours, dependent on the activity of the sample. With similar benefits, U.S. Pat. No. 6,183,978 describes compositions for firefly luciferase assays utilizing myokinase to effect the in-situ generation of AMP to retard the kinetics of the firefly enzyme reaction.

In assays for ATP, the firefly luciferase-luciferin reaction has been utilized to provide the means for the chemiluminescent assay of ATP because of the enzyme's requirement for ATP (Leach, Appl. Biochem. 3: 473-517 (1981)). U.S. Pat. No. 4,833,075 describes the use of immobilized luciferase for the quantitative determination of ATP. U.S. Pat. No. 5,558,986 also describes an ATP assay based on the luciferin-luciferase reaction. In this method a cationic surfactant is utilized to first extract ATP, and a cyclodextrin is then used to neutralize the surfactant, which would otherwise negatively impact the luciferin-luciferase reaction.

U.S. Pat. No. 5,908,751 utilizes pyruvate orthophosphate dikinase in conjunction with a luciferin-luciferase reaction mixture to provide prolonged light output during an ATP assay.

There are drawbacks associated with the use of available assays for firefly luciferase or ATP. Formulations containing high concentrations of thiols are odorous. Thiols also experience auto-oxidation in solution and, as a consequence, are not stable long-term. Commercially available AMP is obtained through yeast fermentation, and may have detracting impurities.

Moreover, in order to facilitate the simultaneous analysis of a number of samples, an improvement in the half-life of emission of the firefly enzyme would be beneficial.

Like the firefly luciferase, Renilla luciferase has found utility in the construction of gene reporter assays. With Renilla, the chemiluminescent substrate employed is a coelenterazine compound (native coelenterazine or a coelenterazine analogue) and there is no requirement for cofactors, or the cosubstrate, ATP. However, oxygen is consumed as a cosubstrate in the Renilla enzymatic reaction. The Renilla enzyme finds use separately in single reporter assays, or more commonly, in concert with firefly luciferase in dual reporter assay formats.

Dual enzyme assays employing the firefly enzyme and Renilla enzyme are disclosed in U.S. Pat. Nos. 5,744,320 and 6,171,809. These methods utilize compositions where the chemiluminescent activity of a first enzyme, i.e., either firefly or Renilla, is assayed, followed by the addition of a second reagent that quenches or partially quenches the activity of the first enzyme, and provides the conditions to allow for the activity of the second enzyme to be assayed in a second step.

### BRIEF SUMMARY OF THE INVENTION

In accordance with the present invention, there is provided an improved composition useful in the chemiluminescent assay of the activity of luciferase, particularly firefly luciferase and Renilla luciferase. The composition provided by the present invention comprises (i) a substrate for a luciferase enzyme, which, upon enzymatic reaction with the luciferase in the presence of any required cosubstrate and/or cofactor, is capable of yielding a chemiluminescent signal, (ii) any cosubstrate and/or cofactor required or beneficial for enzymatic activity of the luciferase, and, as an improvement of the composition, (iii) a water-soluble, organic phosphine-containing compound. It is the presence of this phosphine-containing compound that enables the advantages of the present invention to be realized, particularly the ability to modulate the kinetics of light output from the enzymatic reaction. A preferred water-soluble, organic phosphine-containing compound is Tris(2-carboxyethyl) phosphine (TCEP).

The composition of the present invention can be used in applications designed to quantitate the presence of the enzyme(s) itself, either in single or dual format, or in applications designed to quantitate the presence of a required cosubstrate, such as ATP.

### BRIEF DESCRIPTION OF THE DRAWINGS

FIG. 1 illustrates the time-dependent progression of the measured light output, expressed as observed relative light units (RLU's), from firefly luciferase reactions having no phosphine supplementation or having phosphine supplementation at various concentrations, the phosphine being TCEP.

FIG. 2 illustrates the time-dependent progression of the measured light output from firefly luciferase reactions, expressed as a percentage of the initially measured RLU's, from firefly luciferase reactions having no TCEP supplementation or having TCEP supplementation at various concentrations.

FIG. 3 illustrates the time-dependent progression of the measured light output, expressed as observed RLU's, from thiol-containing firefly luciferase reactions and having no TCEP supplementation or having TCEP supplementation at various concentrations.

FIG. 4 illustrates the time-dependent progression of the measured light output from firefly luciferase reaction mixtures, expressed as a percentage of the initially measured RLU's, from thiol-containing firefly luciferase reaction mixtures having no TCEP supplementation or having TCEP supplementation at various concentrations.

FIG. 5 illustrates the time-dependent progression of the measured light output, expressed as observed RLU's, from firefly luciferase reactions containing no additives, single additives, or combinations of additives.

FIG. 6 illustrates the time-dependent progression of the measured light output, expressed as a percentage of the initially measured RLU's, from firefly luciferase reactions containing no additives, single additives, or combinations of additives.

FIG. 7 illustrates the time-dependent progression of the measured light output, expressed as observed RLU's, from firefly luciferase reactions supplemented with 33.3 μM TCEP and 0-500 μM ATP.

FIG. 8 illustrates the time-dependent progression of the measured light output, expressed as observed RLU's, from firefly luciferase reactions supplemented with 33.3 μM TCEP, 5 mM DTT, and 0-500 μM ATP.

FIG. 9 illustrates the standard curve of measured RLU vs. ATP concentration obtained from the initial reading from firefly luciferase reactions supplemented with 33.3 μM TCEP or supplemented with 33.3 μM TCEP and 5 mM DTT.

FIG. 10 illustrates the time-dependent progression of the measured light output, expressed as observed RLU's, from firefly luciferase reactions containing 33.5 μM TCEP and 238 μM ATP and 0-20 mM DTT.

FIG. 11 illustrates the time-dependent progression of the measured light output, expressed as observed RLU's, from firefly luciferase reactions containing 33.5 μM TCEP and 30 μM ATP and 0-20 mM DTT.

FIG. 12 illustrates the time-dependent progression of the measured light output, expressed as observed RLU's, from Renilla luciferase reactions conducted in the presence or absence of TCEP.

FIG. 13 illustrates the time-dependent progression of the measured light output, expressed as observed RLU's, from firefly luciferase reactions provided by phosphine-containing formulations as compared to a commercially available formulation.

### DETAILED DESCRIPTION OF THE INVENTION

In accordance with the present invention there is provided an improved composition useful in the chemiluminescent assay of the activity of luciferase enzymes, in particular the firefly and Renilla enzymes. The composition comprises (i) a substrate for the luciferase, which, upon enzymatic reaction with the luciferase in the presence of any required cosubstrate and/or cofactor, yields a detectable chemiluminescent signal, (ii) any cosubstrate and/or cofactor required or beneficial for enzymatic activity of the luciferase, and, as an improvement of the composition, a water-soluble, organic phosphine-containing compound.

The presence of the water-soluble, organic phosphine-containing compound in combination with the luciferase substrate is independent of its method of delivery, e.g., it can be packaged separately, as a mixture or in solution. In any event, the phosphine compound and enzyme substrate will be present together in an aqueous solution in the ultimate light-emitting cocktail (i.e., final active assay formulation containing, in addition to the phosphine compound and substrate(s), the enzyme, and, if required or beneficial, any cofactors, as well as other common ingredients).

Useful amounts of the water-soluble, organic phosphine-containing compound in the composition with the substrate are such as to provide a phosphine compound concentration in the light-emitting cocktail of about 0.001-500 mM. In the case of assays employing the firefly luciferase, by varying the concentration of the phosphine compound in the cocktail, e.g., between about 0.001 mM and 500 mM, the kinetics of the light emission arising from the firefly luciferase can be controlled. Control of the kinetics provides the opportunity to select the rate of light decay.

As illustrated hereafter, low concentrations of the phosphine compound in the cocktail (less than about 20 mM) promote high initial enzymatic light output with an essentially constant signal of several minutes (a short sustained burst of light), the light emission then decaying in a linear fashion over the course of time. This is in contrast to formulations without the phosphine compound, where the light output rapidly decays. Higher phosphine compound concentrations (e.g., increasing up to about 100 mM) in the cocktail allow for a more constant steady state output over the entire time course.

The ability to modulate the kinetics of light emission, as above described in assays employing the firefly luciferase, can be important in meeting the analytical requirements of an assay as they relate to available instrumentation and sample handling/throughput issues in the course of conducting luciferase assays. Injector-based assays focus on achieving high sensitivity with minimal concern for processing large numbers of samples (high throughput). Accordingly, these assays are addressed by using low concentrations of the phosphine compound with the associated and consequential initial sustained burst of activity that such formulations provide. Batch processed assays allow for higher throughput but require a less time-sensitive assay condition. These assays are addressed by using higher concentrations of the phosphine compound, allowing the enzymatic activity to be detected in a more steady-state mode.

With respect to specific assays utilizing the instant invention in conjunction with the Renilla luciferase, an important aspect is that the concentrations of the phosphine compound that are generally found useful for the firefly luciferase also can be utilized for the Renilla assay, thus enabling dual enzyme assays to be conducted as well as single assays for this enzyme. An additional aspect is that the phosphine compound allows for a general purpose assay formulation, suitable for the independent measurement of either Renilla luciferase or firefly luciferase activities.

A specific benefit of the phosphine compound in combination with the Renilla luciferase substrate is that it provides for a reduction in the background associated with auto-oxidation of the coelenterazine substrate used in the enzymatic reaction. This leads to improved assay detection sensitivity and formulation stability.

TCEP is a preferred water-soluble, organic phosphine-containing compound, which is capable of modulating luciferase light output to realize the advantages described above. Other unrelated uses of TCEP are shown in U.S. Pat. No. 6,040,150. The compound has substantially no odor and is compatible with the other constituents commonly present in a buffered light-emitting cocktail for firefly luciferase assays. These include ATP, Mg²⁺ and other cofactors, and other additives. While not essential to realizing the advantages provided by the present invention attributable to the presence of the phosphine compound, other ingredients, such as small quantities of thiol-containing compounds, such as DTT and/or CoA, can be present as well in the cocktails. These thiols can provide assay enhancement by increasing the amount of light output.

While TCEP is the preferred water-soluble, organic phosphine-containing compound, other water-soluble, organic compounds having the essential phosphine functionality are considered to be useful. Examples include other organic substituted phosphines containing groups to enhance solubility, such as carboxyl and sulfonic acid groups. Other phosphines also include those similar to TCEP, but wherein the ethyl group is replaced by phenyl, aryl, other alkyl groups, or combinations thereof.

The composition of the present invention is useful in the assay of the activity of luciferase of either of the firefly or Renilla, alone or in a dual enzyme assay format. In addition, the composition can be used in applications designed to quantitate the presence of the enzyme(s) itself, or in applications designed to quantitate the presence of a required cosubstrate, such as ATP.

While both of these assays (for the luciferase enzyme, itself, or for ATP) depend on a chemiluminescent signal arising from the enzymatic reaction from the luciferase/substrate pair, and, thus, assay the activity of a luciferase enzyme, the two types of assays will hereafter be distinguished by being referred to as “Luciferase Assays” and “ATP Assays,” respectively. Luciferase Assays have particular use in gene reporter assays, where the actual activity of the enzyme is itself of interest. ATP Assays have particular use in cell proliferation assays, where ATP levels can be correlated to cell number, as well as in other assays.

The ATP Assay relies on the fact that the firefly luciferase has a requirement for the cosubstrate, ATP, in order to achieve a chemiluminescent signal in the presence of luciferin. Thus, a composition of the present invention for the ATP Assay can include a water-soluble, organic phophine, the firefly enzyme and its luciferin substrate, along with all other necessary components, (e.g., the Mg²⁺ cofactor), except ATP, for chemiluminescent enzymatic activity. Contacting an aqueous solution of this composition with a test solution containing an unknown quantity of ATP generates a chemiluminescent signal that can be quantitated, thus allowing for detection of the ATP analyte. Including a water-soluble, organic phophine in the composition permits modulation of the kinetics of the light output as previously described.

Where the composition of the present invention is to be used in a Luciferase Assay for firefly luciferase, the chemiluminescent substrate is luciferin, which will preferably be supplemented in the composition, when such is provided in a ready-to-use format, with the required cosubstrates, ATP and oxygen, the latter two being considered as cosubstrates, since they are consumed in the enzymatic reaction. The phosphine compound is, of course, present in the composition, and, preferably, so is any required cofactor, such as Mg²⁺, generally added in the form of a chloride or sulfate salt. In a Luciferase Assay for Renilla luciferase, ATP and Mg²⁺ may be omitted from the composition and the substrate is a coelenterazine. Where the composition is to be used in an ATP Assay with firefly luciferase, as indicated previously, the composition contains the phosphine compound and luciferin, but excludes the analyte, ATP. In a ready-to-use format for the ATP Assay the composition contains the luciferase, along with the required cofactor.

In the ready-to-use formats described above, the luciferase substrates and phosphine compound and other ingredients in the composition will generally be dissolved in water. The resulting aqueous solution will typically be buffered to provide pH control, and contain inorganic salts, such as NaCl, to control ionic strength, as well as detergents and other additives.

Assays utilizing the composition of the present invention in aqueous solution are performed under typical atmospheric conditions, which provide a sufficient supply of dissolved oxygen for use as a cosubstrate. Conventional instrumentation employed for Luciferase Assays or ATP Assays may be utilized in using the formulation of the present invention for these assays.

With respect to the Luciferase Assay and ATP Assay, the presence of the phosphine compound avoids the use of high concentrations of odorous thiols and distracting impurities. A further significant feature as mentioned earlier, accompanying use of the present invention is that by varying the phosphine content, light output can be modulated to meet analytical requirements.

An additional advantage with respect to the assay incorporating Renilla luciferase is that the presence of the phosphine compound in combination with the coelenterazine substrate results in the reduction of background associated with the assay. Accordingly, detection sensitivity of the assay is improved. The copresence of the phosphine compound in formulations for Renilla luciferase assays do not detract from the assay procedure and, therefore, enable dual enzyme assay formats.

The following examples further illustrate the invention but, are not intended to limit its scope in any way.

### EXAMPLE 1

This example demonstrates the effect of TCEP inclusion on firefly luciferase reaction.

A common firefly luciferase reaction mix was prepared by combining stock solutions of Tris/Mg buffer, D-luciferin, and ATP. A 900 μl aliquot of this mix consisted of 720 μl of 0.1 M Tris, 10 mM MgCl₂, pH 8.0, 150 μl of 10 mM D-luciferin in 0.1 M Tris, pH 8.0, and 30 μl of 50 mM ATP in 0.1 M Tris, pH 8.0. A series of buffered TCEP preparations were prepared from a neutral pH, 0.5 M TCEP solution (Pierce Chemical Company Product No. 77720) by dilutions with 0.1 M Tris, pH 8.0, as necessary for a TCEP concentration series of 300, 150, 75, 37.5, 18.75, 9.38, 4.69, and 0 mM TCEP. 450 μl of each TCEP concentration were then added separately to 900 μl aliquots of the previously prepared mixture, followed by a final addition of 50 μl of 0.1 M Tris, pH 8.0, to each solution for a final volume of 1.4 ml. The final TCEP concentrations were, therefore, approximately 96, 48, 24, 12, 6, 3, 1.5, and 0 mM and the final Mg, luciferin, and ATP concentrations were 5.14, 1.07, and 1.07 mM, respectively.

Firefly luciferase was diluted from a stock solution (2 μl of 14.7 mg/ml) with 0.1 M Tris, pH 8.0, containing 2 mg/ml bovine serum albumin. 10 μl aliquots containing 181 pg of enzyme were aliquoted into the wells of an opaque white 96-well microplate, after which 125 μl of the reaction mixtures with varying TCEP concentrations were added. The plate was immediately placed into an Orion Luminometer (Berthold Detection Systems), and light output from the plate wells was measured for 15 sequential plate reading cycles, each cycle allowing the wells of the plate to be read with a time interval of approximately 2 minutes.

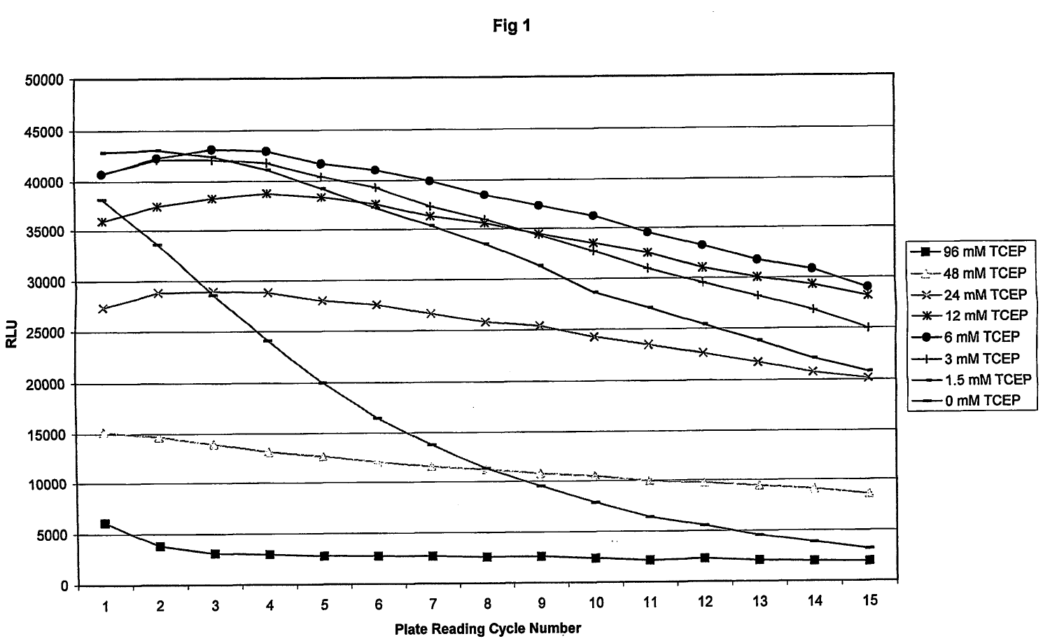

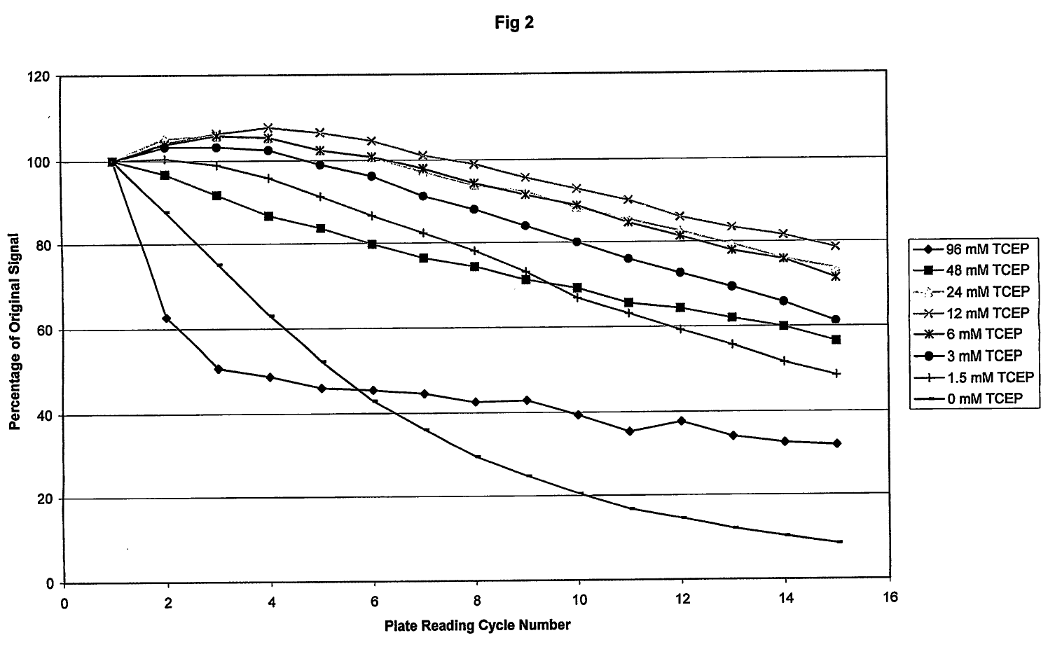

FIGS. 1 and 2 illustrate the results of this experiment. In the absence of enzyme, essentially no signal was observed. FIG. 1 shows the RLU's measured. The control reaction (no TCEP) rapidly decayed over time as shown by the decrease in RLU versus read number. The effect of TCEP was very pronounced even at the lowest TCEP concentration tested. At lower TCEP concentrations (1.5, 3, and 6 mM), the initially observed light output was significantly higher as compared to the control reaction. FIG. 2 shows the retention of original signal versus read number as a percentage of the original signal. All TCEP containing solutions allowed for greater retention of the originally observed light output from the reaction mixtures at the last reading cycle as compared to the control. The control reaction lost greater than 90% of the originally observed light output, whereas the TCEP-containing reaction mixtures retained 32 to 78% of the originally observed signal. With the 96 mM TCEP solution, the light output was more severely decreased with the initial readings. After the fourth cycle, the light output decreased from 49% of the original signal to 32% of the original signal.

### EXAMPLE 2

This example demonstrates the effect of TCEP inclusion on firefly in conjunction with DTT inclusion.

In this experiment, final TCEP concentration was varied from 0-96 mM in the reaction mixture, with the reaction mixture also containing a final concentration of DTT of 5 mM. The conditions for this experiment were identical to that shown in Example 1, but with substitution of the final addition of 50 μl of 0.1 M Tris, pH 8.0, to each solution with a final addition of 50 μl of 50 mM DTT stock solution prepared in 0.1 M Tris, pH 8.0. This experiment was also conducted simultaneously and in the same microwell plate as the experiment of Example 1.

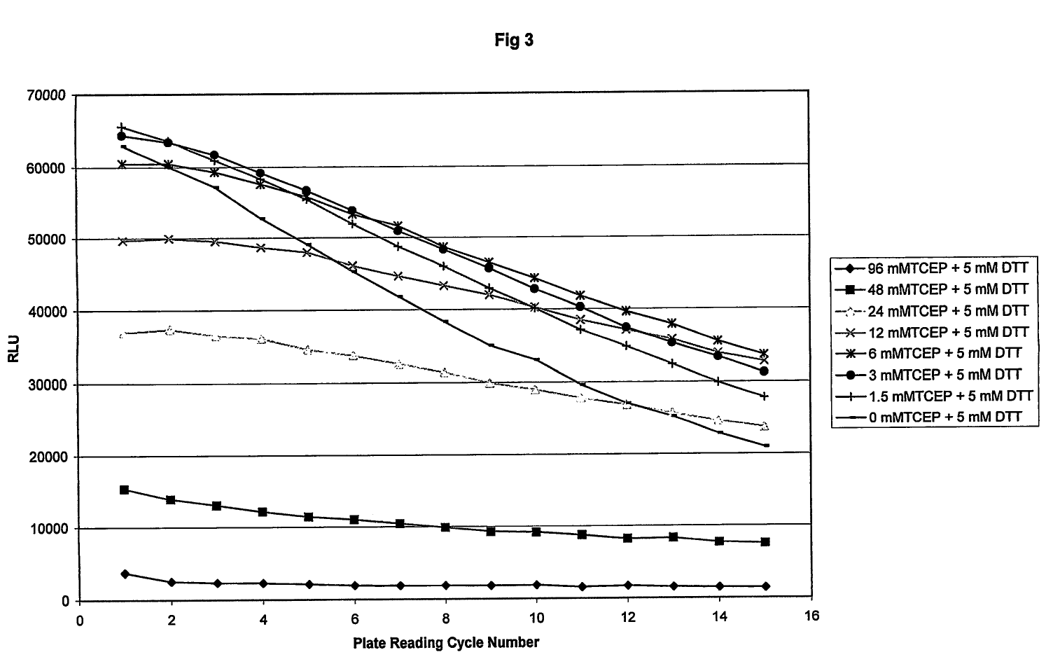

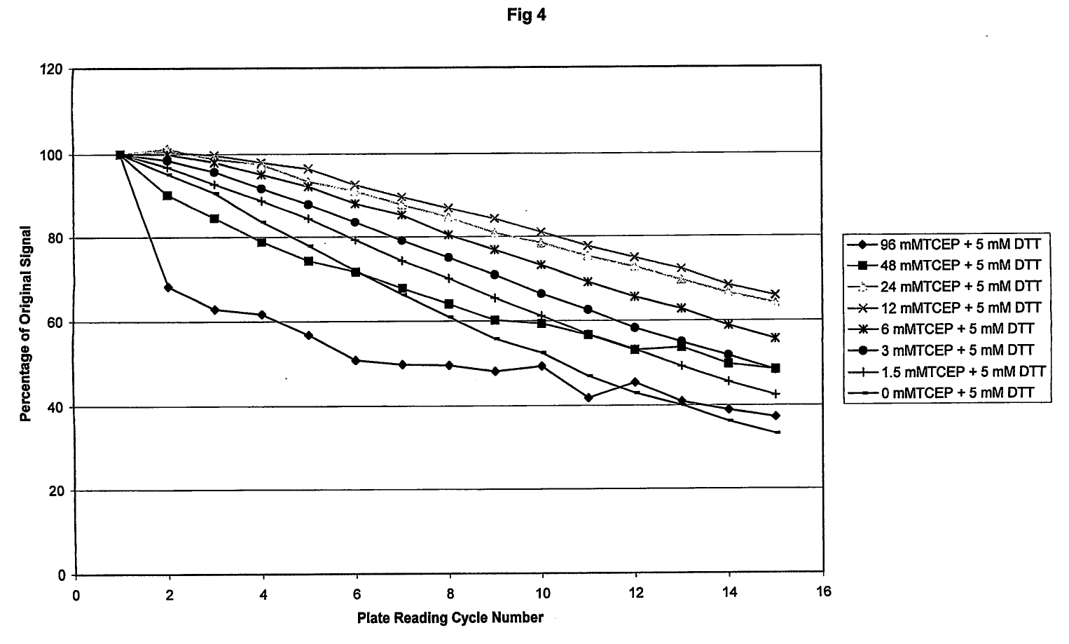

FIGS. 3 and 4 illustrate the results of this experiment. In the absence of enzyme, essentially no signal was observed. FIG. 3 shows the RLU measured. As can be seen from FIG. 3, the initial RLU observed when the reaction mix included either 1.5 or 3 mM TCEP were greater than that given from the control reaction. At the fifteenth reading cycle, greater RLU were observed for the reaction mixtures containing 1.5-24 mM TCEP+DTT as compared to the control reaction mixture containing DTT but without TCEP. FIG. 4 shows the retention of original signal versus read number as a percentage of the original signal. All TCEP-containing solutions allowed for greater retention of the originally observed light output from the reaction mixtures at the last reading cycle as compared to the control.

### EXAMPLE 3

This example demonstrates the effect of substrate additives alone or in combination.

A common firefly luciferase reaction mix was prepared by combining stock solutions of Tris/Mg buffer, D-luciferin, and ATP. A 600 μl aliquot of this mix consisted of 480 μl of 0.1 M Tris, 10 mM MgCl₂, pH 8.0, 85 μl of 10 mM D-luciferin in 0.1 M Tris, pH 8.0, and 17 μl of 50 mM ATP (1889485) in 0.1 M Tris, pH 8.0. Separate aliquots of the reaction mixture were then supplemented with various combinations of additives selected from a 0.5 M TCEP solution, a stock solution of CoA (5 mM CoA in 0.1 M Tris, pH 8.0), or a stock solution of DTT (50 mM DTT in 0.1 M Tris, pH 8.0) and then brought to a final volume of 1 ml with 0.1 M Tris, pH 8.0. In such fashion, the following solutions were obtained: a solution with no additives (None), TCEP (T), TCEP plus CoA (TC), TCEP plus DTT (TD), TCEP plus CoA plus DTT (TCD), CoA (C), CoA plus DTT (CD), and DTT (D or DTT). Where used, the final concentrations of TCEP, CoA, and DTT were 50, 0.5, and 5 mM, respectively. The final Mg²⁺, luciferin, and ATP concentrations were 4.8, 0.85, and 0.85 mM, respectively.

Firefly luciferase was diluted from a stock solution (2 μl of 14.7 mg/ml) with 0.1 M Tris, pH 8.0, containing 2 mg/ml bovine serum albumin. 10 μl aliquots containing 181 pg of enzyme were aliquoted into the wells of a white opaque 96-well microplate, after which 125 μl of the various reactions were added. The plate was immediately placed into an Orion Luminometer, and light output from the plate wells was measured with sequential plate reading cycles over a one hour time frame, each cycle allowing the wells of the plate to be read with a time interval of approximately 140 seconds.

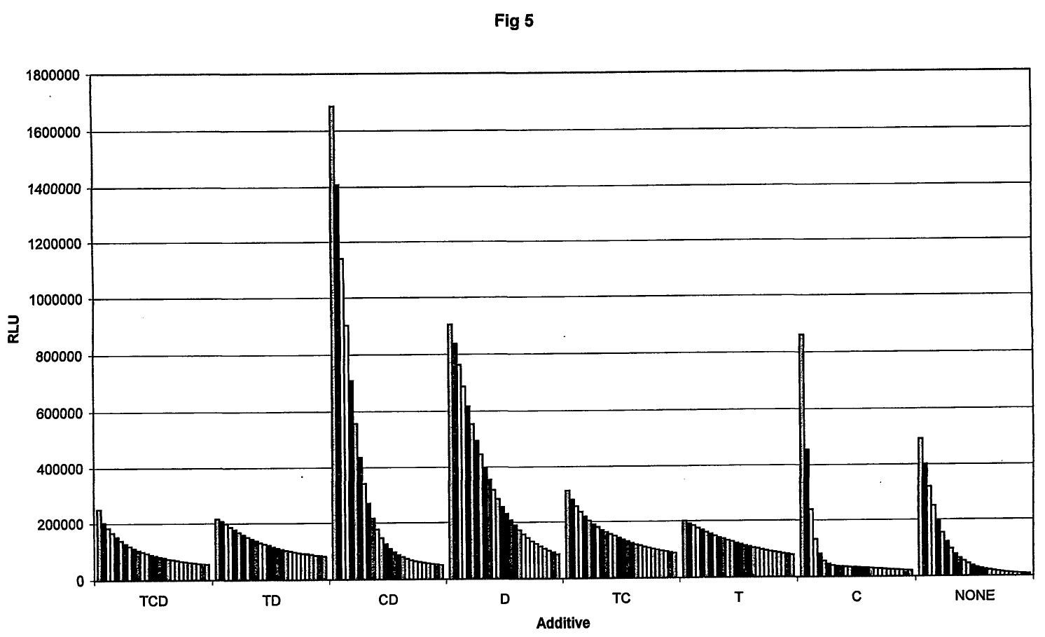

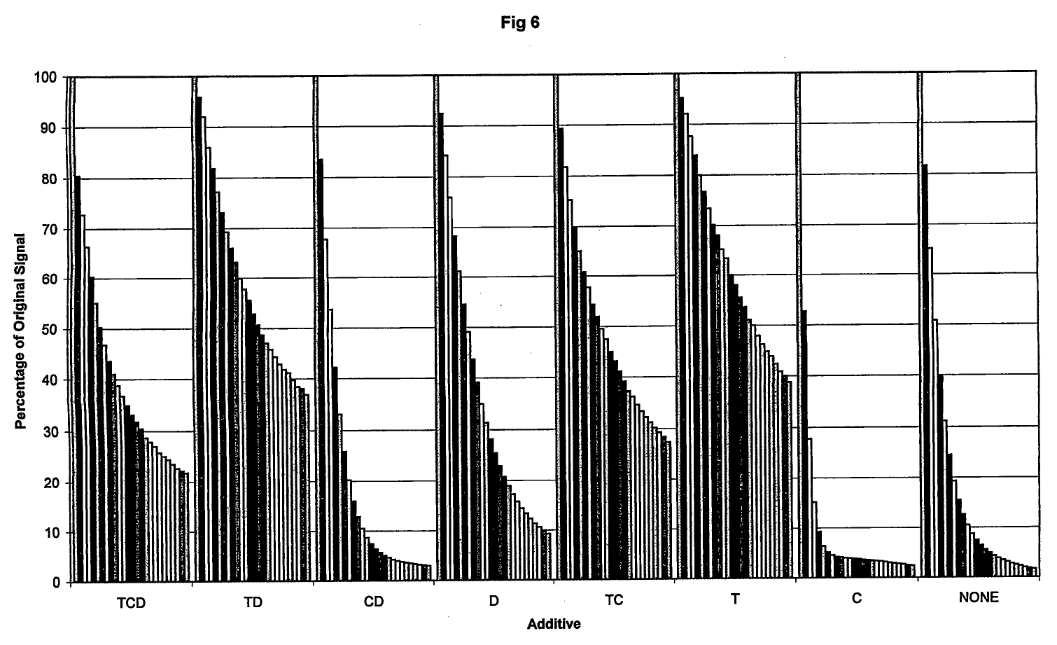

FIGS. 5 and 6 illustrate the results of this experiment. In the absence of enzyme, essentially no signal was observed. FIG. 5. shows the RLU measured. Inclusion of CoA, DTT, or the combination of CoA plus DTT increased the initial light output observed as compared to the no addition control, but the levels of light output rapidly decreased. The inclusion of CoA, DTT, or CoA plus DTT into solutions also supplemented with TCEP increased the initial amount of light output observed as compared to the use of only TCEP. All of the TCEP containing formulations gave solutions with greater half-lives of emission as compared to those lacking TCEP. The light output did not decay as rapidly when the enzyme was assayed in the presence of TCEP as compared to the absence of TCEP. This is readily observed when the data are graphed as a function of percentage of the original light output observed, and is shown in FIG. 6. The point at which the first half-life decay occurred was two-fold longer in both of the cases of the TCD solution (vs. CD solution) and the TD solution (vs. D solution) and was 10-fold longer in the case of the TC solution (vs. C solution).

### EXAMPLE 4

This example demonstrates ATP assay utilizing TCEP in a firefly luciferin-luciferase reaction.

A common solution was prepared by combining stock solutions of Tris/Mg buffer, D-luciferin, and TCEP. A 2.25 ml aliquot of this solution contained 1.2 ml of 0.1 M Tris, 10 mM MgCl2, pH 8.0, 18.75 μl of 10 mM D-luciferin in 0.1 M Tris, pH 8.0, 165 μl of 0.5 M TCEP, with the remaining volume being 0.1 M Tris, pH 8.0. 0.5 ml of this mixture was removed and to it was added 11 μl of an ATP stock solution (50 mM ATP in 0.1 M Tris, pH 8.0) and 39 μl of 0.1 M Tris, pH 8.0; the remaining 1.75 ml of mixture received 175 μl of 0.1 M Tris, pH 8.0. The ATP-containing aliquot was then serially diluted with the non-ATP-containing mixture to yield a final ATP concentration series of 500, 250, 125, 62.5, 31.25, 15.625, and 0 μM. The non-ATP-containing mixture served as the control for zero ATP concentration. In this series of reaction mixtures with varying concentrations of ATP, the final luciferin, magnesium, and TCEP concentrations were 75.8 μM luciferin, 4.8 mM magnesium, and 33.3 mM TCEP.

In similar fashion, another ATP concentration series was prepared, but with the formulation also containing a final DTT concentration of 5 mM DTT.

Firefly luciferase was diluted from a stock solution (2 μl of 14.7 mg/ml) with 0.1 M Tris, pH 8.0 containing 2 mg/ml bovine serum albumin. 10 μl aliquots containing 181 pg of enzyme were aliquoted into the wells of an opaque white 96-well microplate, after which 100 μl of the reaction mixtures with varying ATP concentrations were added. The plate was immediately placed into an Orion Luminometer and light output from the wells was measured with sequential plate reading cycles, each cycle allowing the wells of the plate to be read with a time interval of approximately 2.25 minutes. In the absence of enzyme, essentially no light output was observed.

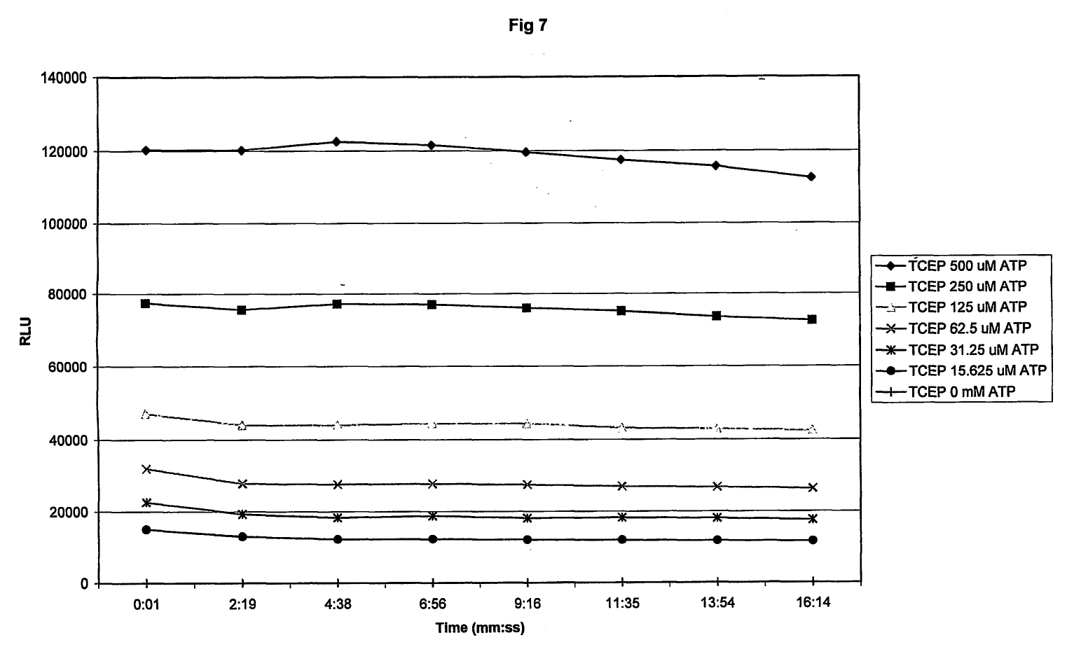

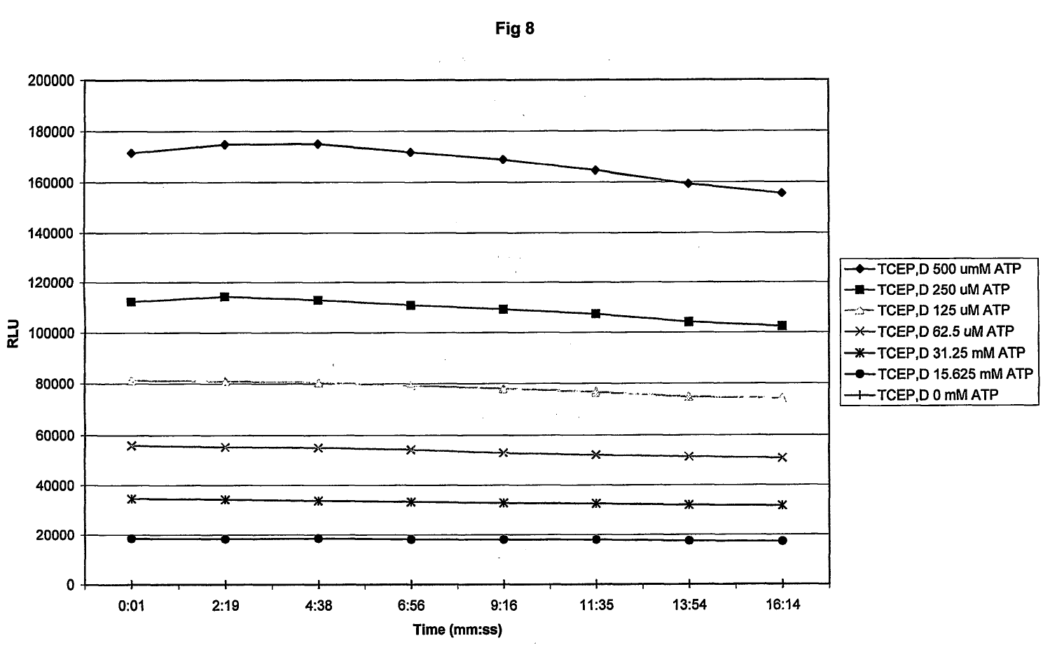

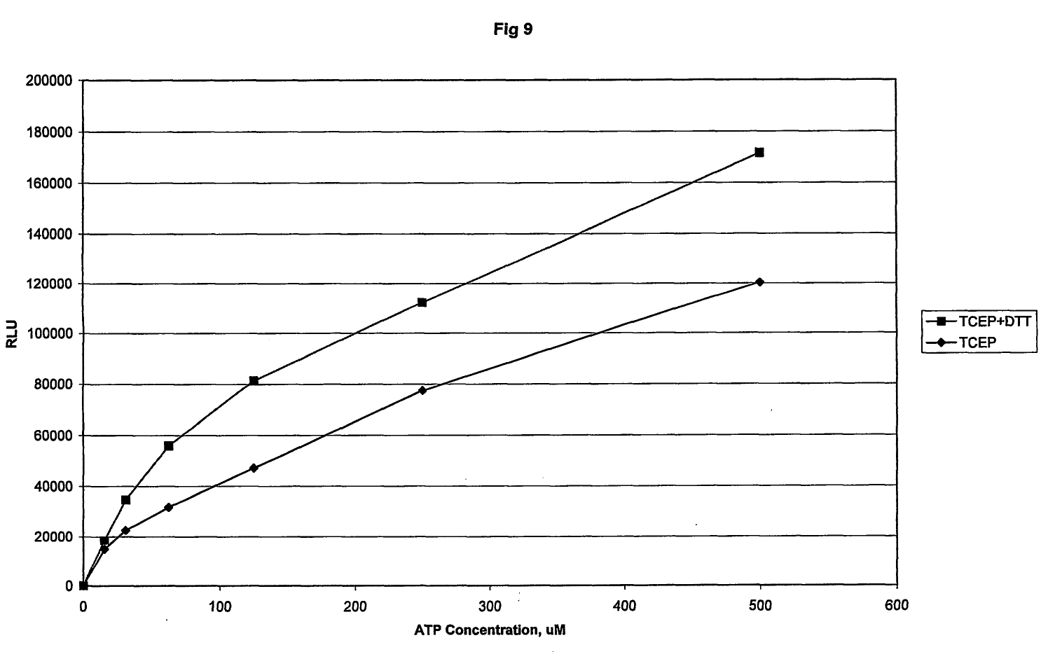

FIG. 7 and FIG. 8 illustrate the results of these experiments for the first 8 sequential readings. Both the TCEP reaction mixtures (FIG. 7) and the TCEP/DTT reaction mixtures (FIG. 8) yielded prolonged light output without rapid decay of the original signal. Also, the light output increased with increasing ATP concentration. The RLUs were elevated when 5 mM DTT was included in the reaction mixture. FIG. 9 illustrates the results of these experiments with the first reading cycle by plotting RLU vs. ATP concentration. A standard curve for ATP concentration is thereby obtained when the invention is practiced as an ATP assay.

### EXAMPLE 5

This example demonstrates the role of thiols in TCEP-mediated firefly reactions.

A common solution was prepared by combining stock solutions of Tris/Mg buffer, D-luciferin, TCEP, and ATP. This solution was prepared by combining 12 ml of 0.1 M Tris, 10 mM MgCl₂, pH 8.0, 187.5 μl of 10 mM D-luciferin in 0.1 M Tris, pH 8.0, 1.667 of 0.5 M TCEP, and 30 μl of an ATP stock solution (50 mM ATP in 0.1 M Tris, pH 8.0) and 1.02 ml of 0.1 M Tris, pH 8.0. A 150 μl aliquot of this solution was added to 100 μl of 50 mM DTT stock solution (50 mM DTT in 0.1 M Tris, pH 8.0) for a final DTT concentration of 20 mM, and another 1.35 ml aliquot of the solution received 0.9 ml 0.1 M Tris, pH 8.0. The 20 mM DTT reaction solution was further diluted with the second aliquot to give a final DTT concentration series of 20, 10, 5, 2.5, 1.25, 0.6, 0.3, 0.15, 0.02, and 0 mM DTT. In this series of reaction mixtures with varying concentrations of DTT, the final luciferin, magnesium, and TCEP concentrations were 75.5 μM luciferin, 4.8 mM magnesium, and 33.5 mM TCEP and 238 μM ATP. Other reaction mixtures with varying DTT concentrations were also prepared in like fashion, but with a final ATP concentration held constant at either 59.5, 29.75 or 14.875 μM, which was achieved by further dilution of the ATP solution with 0.1 M Tris, pH 8.

Firefly luciferase was diluted from a stock solution (2 μl of 14.7 mg/ml) with 0.1 M Tris, pH 8.0, containing 2 mg/ml bovine serum albumin. 10 μl aliquots containing 181 pg of enzyme were aliquoted into the wells of an opaque white 96-well microplate, after which 100 μl of the reaction mixtures with varying DTT concentrations were added. The plate was immediately placed into an Orion Luminometer, and light output from the wells was measured with sequential plate reading cycles, each cycle allowing the wells of the plate to be read with a time interval of approximately 2.25 minutes.

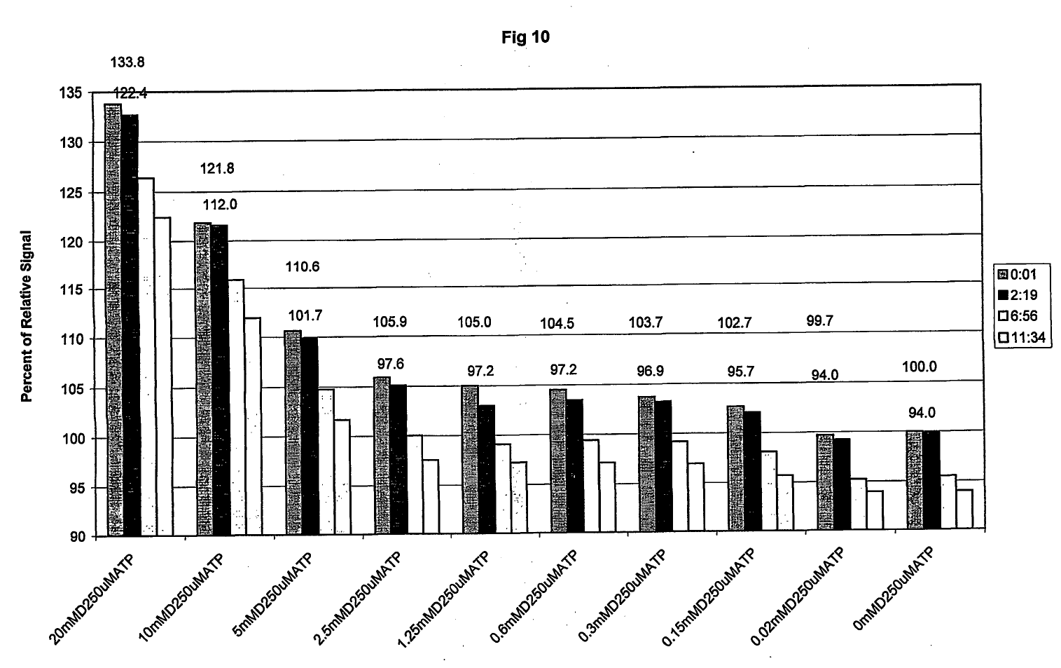

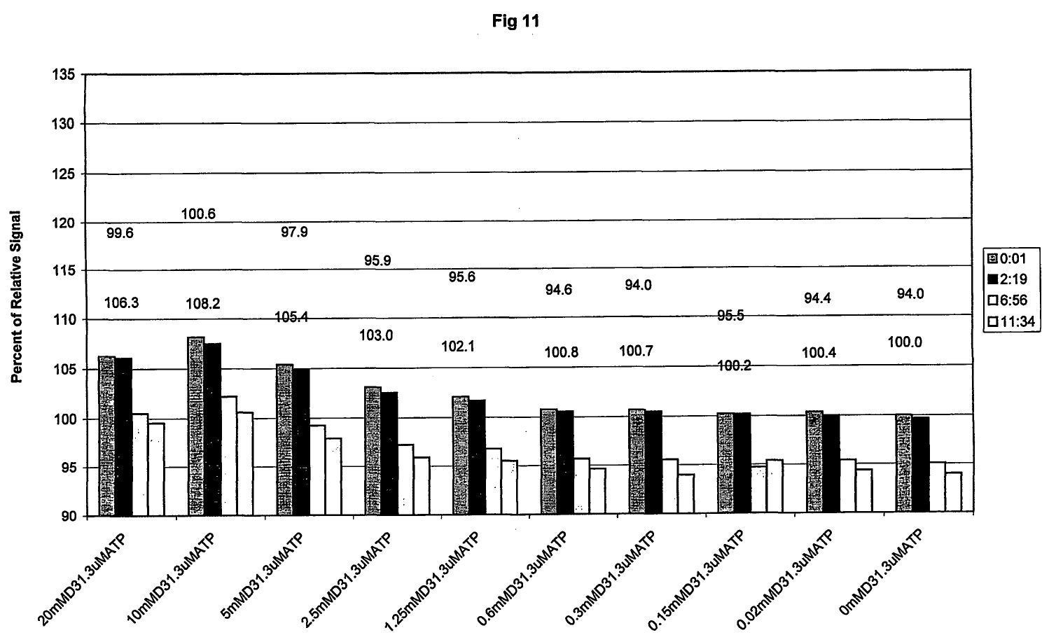

FIGS. 10 and 11 illustrate the results of this experiment. FIG. 10 illustrates the results obtained where the final ATP concentration of the reaction mixture was 238 μM, and FIG. 11 illustrates the results obtained where the final ATP concentration of the reaction mixture was 30 μM. Both graphs relate the observed RLU at each reading cycle expressed as a relative percentage of the signal obtained with the well receiving no DTT supplementation at the first reading cycle, with this signal being assigned 100%.

As can be seen in both graphs, the signal increased with increasing DTT concentration. The initial RLU observed during the first cycle increased about 34% at 20 mM DTT when the ATP concentration was 238 μM. The initial RLU observed during the first cycle increased about 8% at 10 mM DTT when the ATP concentration was 30 μM. At 30 μM ATP, the effect of DTT was saturating at 20 mM, as evidenced by the plateaued increase in observed RLU.

Information on the mechanism of thiol inclusion in TCEP-containing reaction mixtures can be ascertained from observing the decrease as a percentage of the original signal at each DTT concentration. At both 250 and 30 μM ATP concentration without added DTT, the signal decreased in both cases about 6% in the time frame from the first reading cycle to reading cycle that was 11 minutes 34 seconds later. In the presence of increasing thiol concentration, the loss of signal was more substantial, and this effect was more pronounced at higher ATP concentrations. At 20 mM DTT and 250 μM ATP, the enzyme was outputting 8.5% less light after 11.5 minutes as compared to the initial reading. Thus, in TCEP-containing reaction mixtures, it is concluded that thiols do not contribute to improving enzyme stability during catalysis. Were thiols contributing to improving the enzyme stability during catalysis, the percentage drop in the original signal as compared to the 11.5 minute signal would have been increased with increasing thiol concentration.

### EXAMPLE 6

This example demonstrates the use of TCEP in a Renilla assay.

In order to ascertain whether or not TCEP would interfere with a Renilla assay, an experiment was conducted utilizing a Renilla assay mixture, with or without TCEP supplementation. A Renilla assay mixture was prepared by combining 480 μl of 0.1 M Tris, 10 mM MgCl₂, 1 mM EDTA, pH 8.0, with 437.7 μl of 0.1 M Tris, pH 8.0, and 16.7 μl of a coelenterazine stock solution (59 μM coelenterazine in denatured ethanol prepared from Molecular Probes Catalog number C-6777). This solution was split into 2 aliquots of 466.7 μl, and one aliquot received 33.3 μl of 0.5 M TCEP and the other aliquot received 33.3 μl 0.1 M Tris, pH 8.0.

Renilla luciferase purchased from Chemicon (Catalog number 4400) was diluted with 1 ml phosphate-buffered saline (10 mM sodium phosphate, 150 mM NaCl, pH 7.2) and stored frozen at −80° C. in 10 μl aliquots, each aliquot containing 100 ng enzyme. An aliquot was removed and diluted with 0.1 M Tris, pH 8.0, for a concentration series of 166, 55, 18.5, and 0 pg/10 μl. 10 μl of each enzyme concentration were pipetted in duplicate to the wells of a white opaque 96-well microplate, and 100 μl of either of the TCEP containing solution or the solution without TCEP was pipetted into the wells to initiate the reaction. The plate was immediately placed into an Orion Luminometer, and light output from the wells was measured with sequential plate reading cycles.

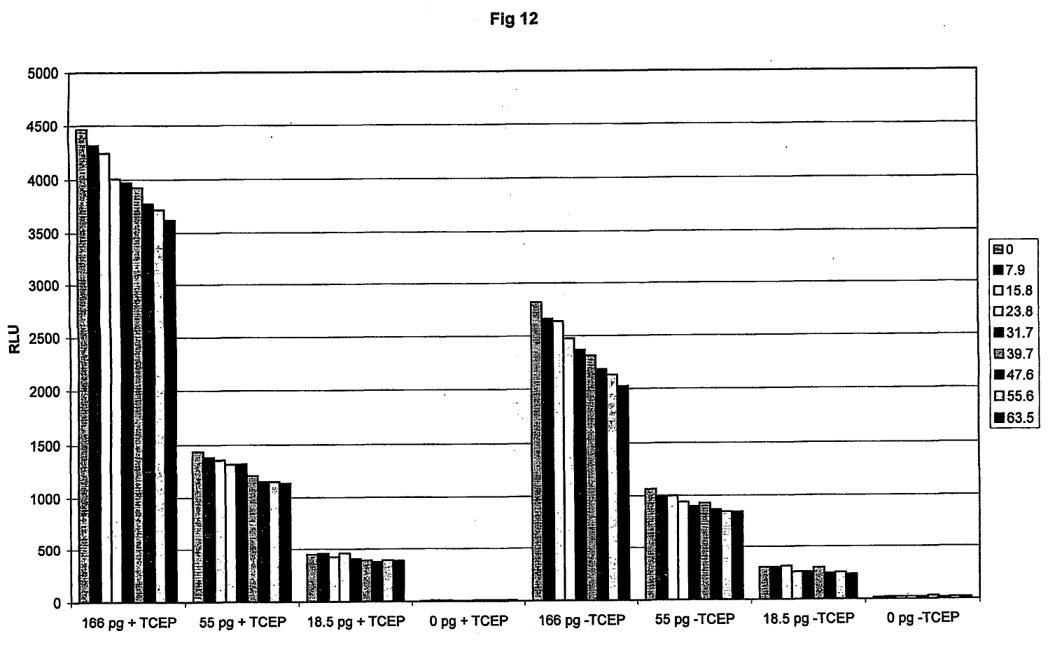

FIG. 12 illustrates the results of the experiment for the first initial reading cycles from 0-63.5 seconds (interval time approximately 8 seconds). The TCEP-containing solution allowed for the reaction to proceed. In the presence of TCEP, the background (no added enzyme) was reduced 50% as compared to the absence of TCEP. With additional incubation time, the light output rapidly diminished in both the TCEP and no TCEP reaction mixtures at approximately equal rates.

### EXAMPLE 7

This example demonstrates the sequential assay of firefly and Renilla luciferases.

A firefly luciferase reaction mix is prepared by combining stock solutions of Tris/Mg buffer, D-luciferin, and ATP. A 900 μl aliquot of this mix consisted of 720 μl of 0.1 M Tris, 10 mM MgCl₂, pH 8.0, 150 μl of 10 mM D-luciferin in 0.1 M Tris, pH 8.0, and 150 μl of 50 mM ATP (1889485) in 0.1 M Tris, pH 8.0. A buffered TCEP preparation is prepared from a neutral pH, 0.5 M TCEP solution by dilution with 0.1 M Tris, pH 8.0, for a TCEP concentration of 75 mM. 450 μl of the TCEP solution are then added to the 900 μl aliquot of the previously prepared mix, followed by a final addition of 50 μl of 0.1 M Tris, pH 8.0, for a final volume of 1.4 ml. The final TCEP concentration is, therefore, approximately 24 mM and the final Mg²⁺, luciferin, and ATP concentrations are 5.1, 1.07, and 5.35 mM, respectively.

Firefly luciferase is diluted from a stock solution (2 μl of 14.7 mg/ml) with 0.1 M Tris, pH 8.0, containing 2 mg/ml bovine serum albumin. 10 μl aliquots containing 181 pg of enzyme are aliquoted into the wells of an opaque white 96-well microplate, after which 125 μl of the above reaction mixture are added. The plate is then immediately placed into an Orion Luminometer (Berthold Detection Systems), and light output from the plate wells is measured. High levels of light output from the firefly luciferase are observed. Upon addition of 125 μl of a solution comprising 4 μM coelenterazine, 50 mM EDTA, 0.1 M Tris, pH 7.0, the light output is substantially and quickly quenched. Upon addition of a dilution of purified Renilla luciferase enzyme, activity resulting from enzyme turnover of the substrate is observed and the levels of light output are concentration-dependent with respect to Renilla enzyme concentration.

### EXAMPLE 8

This example demonstrates a comparison of phosphine-containing formulations to commercial formulations.

Six variations of the phosphine-containing formulations of the present invention were prepared and assessed for relative performance to a commercially available formulation. Each of these six phosphine-containing formulations contained a common final concentration of the following components: 33.3 mM TCEP, 75.8 μM D-luciferin, 4.5 mM magnesium (as Mg²⁺), 0.1 M Tris, pH 8.0. Phosphine Formulation 1, 2, 3, 4, 5, and 6, further contained 500 μM ATP and 5 mM DTT; 500 μM ATP; 250 μM ATP and 5 mM DTT; 125 μM ATP and 5 mM DTT; 250 μM ATP; and 125 μM ATP, respectively. The commercial formulation was prepared according to the manufacturer's instructions.

Firefly luciferase was diluted from a stock solution (2 μL1 of 14.7 mg/ml) with 0.1 M Tris, pH 8.0, containing 2 mg/ml bovine serum albumin. 10 μl aliquots containing 181 pg of enzyme were aliquoted into the wells of an opaque white 96-well microplate after which 100 μl of the various phosphine-containing formulations and the commercial formulation were added. The plate was immediately placed into an Orion Luminometer, and light output from the wells was measured with sequential plate reading cycles, each cycle allowing the wells of the plate to be read with a time interval of approximately 2.25 minutes. In the absence of enzyme, essentially no light output was observed in all cases.

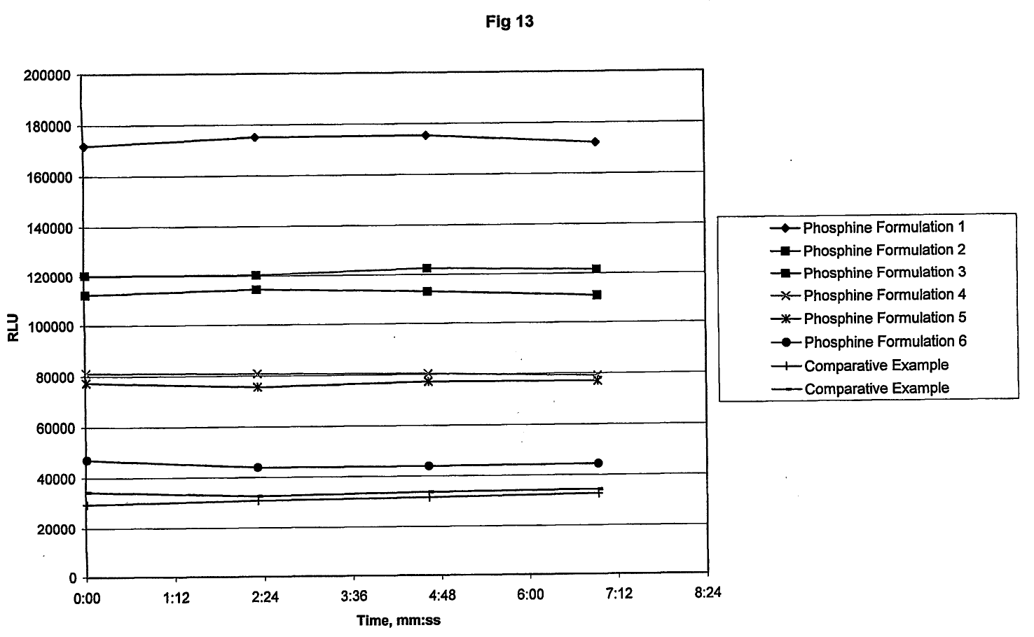

FIG. 13 illustrates the results of these experiments for the first 4 sequential readings. The phosphine-containing solutions provided for stable light output from the enzymatic reaction in similar fashion to the commercial formulation, but in all cases the phosphine-containing formulations provided for greater light output from the enzymatic reaction as compared to the commercial formulation. The phosphine-containing formulations provided for stable light output in the absence of the thiol-reagent DTT, indicating that the thiol-containing reagent did not contribute to the stability of the light output from the enzymatic reaction using formulations containing the phosphine compound. Supplementation of phosphine-containing formulations with the thiol-containing reagent enhanced the observed light output from the enzymatic reaction as compared to its absence.

All references, including publications, patent applications, (including U.S. Provisional Application No. 60/332,843, to which this application claims priority), and patents, cited herein are hereby incorporated by reference to the same extent as if each reference were individually and specifically indicated to be incorporated by reference and were set forth in its entirety herein.

While the invention has been described with emphasis upon preferred embodiments, it will be obvious to those skilled in the art that variations of the preferred embodiments may be used and that it is intended that the invention may be practiced otherwise than as specifically described herein. In particular, but without limiting the foregoing, the Examples illustrate the use of firefly luciferase as an ATP-dependent luciferase. However, the invention is not limited with respect to a specific firefly luciferase and, accordingly, may include recombinant variants of the enzyme, or native firefly luciferase, or functional equivalents of native firefly luciferase. Accordingly, this invention includes all modifications encompassed within the spirit and scope of the invention as defined by the following claims.

1. A composition useful in the chemiluminescent assay of the activity of a luciferase, which composition comprises (i) a substrate for the luciferase, which, upon enzymatic reaction with the luciferase in the presence of any required cosubstrate and/or cofactor, yields a detectable chemiluminescent signal, (ii) any cosubstrate and/or cofactor required or beneficial for enzymatic activity of the luciferase, and, as an improvement of the composition, (iii) a water-soluble, organic phosphine-containing compound.

2. The composition of claim 1, wherein the luciferase is selected from the group consisting of firefly luciferase, Renilla luciferase, or a combination thereof.

3. The composition of claim 1, wherein the water-soluble, organic phosphine-containing compound is Tris(2-carboxyethyl) phosphine (TCEP).

4. The composition of claim 2, wherein the water-soluble, organic phosphine-containing compound is TCEP.

5. The composition of claim 1, wherein the enzyme is firefly luciferase and the substrate is the D-isomer of luciferin.

6. The composition of claim 5, wherein the water-soluble, organic phosphine-containing compound is TCEP.

7. The composition of claim 5, wherein the cofactors is Mg²⁺.

8. The composition of claim 6, wherein the cofactor is Mg²⁺.

9. The composition of claim 7, wherein the cosubstrate is ATP.

10. The composition of claim 8, wherein the cosubstrate is ATP.

11. The composition of claim 1, wherein the enzyme is Renilla luciferase and the substrate is a coelenterazine compound.

12. The composition of claim 11, wherein the water-soluble, organic phosphine-containing compound is TCEP.

13. The composition of claim 1, which further comprises a thiol compound.

14. The composition of claim 13, wherein the thiol compound is dithiothreitol (DTT), Coenzyme A (CoA), or a combination thereof.

15. An aqueous solution comprising the composition of claim 1.

16. An aqueous solution comprising the composition of claim 6.

17. In a chemiluminescent assay of the activity of a luciferase, which assay comprises: (i) reacting the luciferase with a substrate in the presence of any required cosubstrate and/or cofactor, wherein the substrate yields a detectable chemiluminescent signal upon enzymatic reaction with the luciferase, and (ii) detecting the chemiluminescent signal, the improvements comprising reacting the luciferase with the substrate in the presence of a water-soluble, organic phosphine-containing compound.

18. The assay of claim 17, which further comprises (iii) quantitating the amount of luciferase.

19. The assay of claim 17, which further comprises (iii) quantitating the amounts of a required cosubstrate.
